(19) United States
(12) Patent Application Publication
(10) Pub. No.: US 2021/0340625 A1
(43) Pub. Date: Nov. 4, 2021

(54) tRNA-DERIVED FRAGMENTS AS BIOMARKERS FOR PARKINSON'S DISEASE

(71) Applicant: THOMAS JEFFERSON UNIVERSITY, Philadelphia, PA (US)

(72) Inventors: Isidore Rigoutsos, Daejeon, NY (US); Rogan MAGEE, Philadelphia, PA (US); Eric LONDIN, Philadelphia, PA (US)

(21) Appl. No.: 17/283,941

(22) PCT Filed: Oct. 10, 2019
(86) PCT No.: PCT/US2019/055648

§ 371 Date: Apr. 8, 2021

Related U.S. Application Data
(60) Provisional application No. 62/744,009, filed on Oct. 10, 2018.

(51) Int. Cl. C12Q 1/6883

(52) U.S. Cl. CPC C12Q 1/6883; C12Q 2600/158

(57) ABSTRACT

The present invention includes a method for analyzing tRNA-derived fragments. In one aspect, the present invention includes a method of identifying a subject in need of therapeutic intervention to treat a disease or condition, disease recurrence, or disease progression comprising characterizing the identity of tRNA fragments. The invention also includes diagnosing, identifying or monitoring a disease or condition, and a method for identifying tRNA fragments. The invention also includes diagnosing, identifying or monitoring Parkinson's disease in a subject in need thereof by characterizing the identity of tRNA fragments.

### CROSS-REFERENCE TO RELATED APPLICATIONS

This application claims priority under 35 U.S.C. § 119(e) to U.S. Provisional Patent Application No. 62/744,009, filed Oct. 10, 2018, which application is incorporated herein by reference in its entirety.

### BACKGROUND OF THE INVENTION

Transfer RNA (tRNA) molecules serve as a physical link between the snRNA and the amino acid sequence of proteins. The tRNA carries an amino acid to the protein synthetic machinery of a cell (ribosome) as directed by a 3-nucleotide sequence (codon) in a messenger RNA (mRNA), thereby serving as necessary components of translation, the biological synthesis of new proteins in accordance with the genetic code.

tRNA halves (tRHs) are produced in response to cellular stress and represent the first reported category of tRNA-derived fragments (CRFs). There are two variants of tRHs: 5′-tRNA halves (5′-tRH) and 3′-tRNA halves (3′-tRH). The endonuclease Angiogenin (ANG) has been shown to govern the production of tRHs from mature tRNAs, in several species and contexts. Subsequent work revealed two more categories that overlap the mature tRNA: the 5′-tRFs, whose 5′ termini are the 5′ ends of the mature tRNA, and the 3′-CRFs, whose 3′ termini are the 3′ end of the mature tRNA. Another category of tRFs are known as “internal tRFs” (i-tRFs); i-tRFs can begin and end anywhere along the mature tRNA sequence.

Functionally, tRFs have been linked to a variety of specific regulatory roles. Some tRFs act through miRNA pathway mechanisms, whereas other directly interact with cellular proteins such as cytochrome C. tRFs can also act as decoys for RNA-binding proteins, resulting in stabilization of oncogenic transcripts. Yet other tRFs directly interfere with translation, or disrupt formation of the multi-synthetase complex or ribosome biogenesis. tRFs have also been shown to control retrotransposon and reverse transcriptase activity in animals and plants.

Despite all the previously-reported links of different tRFs to different contexts there still exists an unmet need in the art to identify, characterize, quantify and ascertain the role of tRFs in diseased and healthy cells, as well as leverage information about these links for diagnosis and treatment. This invention contributes to the efforts to address these needs.

### BRIEF SUMMARY OF THE INVENTION

In one aspect, the invention provides a method of identifying a subject in need of therapeutic intervention to treat a disease, condition, disease recurrence, or disease progression. In certain embodiments, the method comprises isolating fragments derived from tRNAs (tRFs) from a sample obtained from the subject; and characterizing the tRFs and their relative abundance in the sample to identify a signature, wherein when the signature is indicative of a diagnosis of the disease, a treatment of the subject is recommended.

In another aspect, the invention comprises a method of diagnosing, identifying or monitoring Parkinson's Disease (PD) in a subject in need thereof. In certain embodiments, the method comprises isolating tRFs from a cell obtained from the subject, quantifying the tRFs using a panel of oligonucleotides engineered to detect tRFs, or another method; analyzing levels of the tRFs present in the cell; wherein a differential in the level of measured tRFs as compared to a reference is indicative of a diagnosis or identification of PD in the subject; and providing a treatment regimen to the subject dependent on the differential in the level of measured tRFs as compared to the reference.

In yet another aspect, the invention comprises a method of identifying a subject at risk for developing Parkinson's Disease (PD) or in need of therapeutic intervention to treat Parkinson's disease (PD). The method comprises steps of isolating fragments of tRFs from a sample obtained from the subject; quantifying the tRFs using a panel of oligonucleotides engineered to detect tRFs, or another method; and characterizing the tRFs and their relative abundance in the sample to identify a signature, wherein when the signature is indicative of a prognosis for developing PD or a diagnosis for a PD, a treatment of the subject is recommended.

In yet another aspect, the invention provides a kit for high-throughput analysis of tRFs fragments in a sample from a subject in need thereof, the kit comprising a collection of specially-designed qPCR assays for quantitating tRFs, or a panel of engineered oligonucleotides capable of hybridizing tRFs, or another quantification method.

In certain embodiments, the tRFs are selected from the group consisting of 3′-tRFs, 3′-tRFs, 5′-tRFs, and i-tRFs from a mitochondrion (MT). In certain embodiments, the tRFs are selected from the group consisting of 3′-tRFs, 3′-tRHs, 5′-tRFs, 5′-tRHs, and i-tRFs a nucleus (Nuc).

In certain embodiments, the sample is isolated from a cell, a tissue, an extracellular vesicle, or a body fluid obtained from the subject. In certain embodiments, the body fluid, the extracellular vesicle, the tissue or the cell is selected from the group consisting of bile, blood serum, plasma, cerebrospinal fluid, and prefrontal cortex.

In certain embodiments, isolating the tRFs comprises isolating tRFs fragments with a length in the range of about 10 nucleotides to about 70 nucleotides. In certain embodiments, the signature comprises at least one sequence selected from the group consisting of SEQ ID NO: 53135-SEQ ID NO: 63850.

In certain embodiments, the tRFs and their relative abundance vary between the normal state as compared to disease state or condition. In certain embodiments, the tRFs and their relative abundance vary depending on sex of the subject. in certain embodiments, the disease or condition, disease recurrence, or disease progression is a brain disease. In certain embodiments, the brain disease is genetically predisposed. In certain embodiments, the brain disease is Parkinson's Disease.

In certain embodiments, characterizing the tRFs comprises at least one assessment selected from the group consisting of sequencing tRFs, measuring overall abundance of a tRF mapped to the genome, measuring a relative abundance of a tRF to a reference, assessing a length of a tRF, identifying starting and ending points of a tRF, identifying genomic origin of a tRF, and identifying a terminal modification of a tRF.

In certain embodiments, the tRFs and their relative abundance vary between the normal state subject as compared to a subject at a risk of or suffering from PD. In certain embodiments, the tRFs and their relative abundance compared to control subjects differ between subjects suffering from PD with dementia and subjects suffering from PD without dementia.

In certain embodiments, the tRFs and their relative abundance vary with the body fluid, extracellular vesicle, cell or tissue sample. In certain embodiments, the tRFs and their relative abundance vary in the prefrontal cortex samples from the normal state subjects, the subjects suffering from PD with dementia, and the subjects suffering from PD without dementia.

In certain embodiments, the tRFs and their relative abundance vary in the cerebrospinal fluid samples from the normal state subjects, the subjects suffering from PD with dementia, and the subjects suffering from PD without dementia.

In certain embodiments, the tRFs and their relative abundance vary in the serum samples from the normal state subjects, the subjects suffering from PD with dementia, and the subjects suffering from PD without dementia.

In certain embodiments, the subject is a human,

### BRIEF DESCRIPTION OF THE DRAWINGS

The following detailed description of preferred embodiments of the invention will be better understood when read in conjunction with the appended drawings. For the purpose of illustrating the invention, there are examples shown in the drawings embodiments of which are presently preferred. It should be understood, however, that the invention is not limited to the precise arrangements and instrumentalities of the embodiments shown in the drawings.

FIGS. 1A-1F: tRFs identified in PFC, CSF and serum samples. The MINTmap and Threshold-sect algorithms were used to identify the set of tRFs recovered from all PFC samples, all CSF samples, and all SER samples. (FIG. 1A) The Venn diagram describes the overlap of tRFs discovered in these three collections. (FIG. 1B) The overlap shown in (FIG. 1A) was recomputed after filtering out any tRFs that are contained in the most recent Release 2.0 of MINTbase. Use oft tests allowed the identification of tRFs that are differentially abundant (DA) between control and Parkinson's disease samples in PFC (prefrontal cortex), CSF (cerebrospinal fluid), and SER (serum) samples. DA tRFs are those with a difference in means per a t test with p-value≤0.05. The shown Venn diagrams capture the overlap between tRFs identified in each context (FIGS. 1C-1F). Each panel describes the overlap of DA tRFs when splitting samples by patient sex (male or female) and sample tissue type (PFC, CSF, or SER), as indicated. Green circles represent PFC samples. Gold circles represent CSF samples. Red circles represent SER. samples.

FIGS. 2A-2F: DA tRFs in PFC, CSF, and SER samples. The DA tRFs were identified as described herein. The results showing fold changes and p-values, in combination with a breakdown by tRF type were plotted. Specifically, the logₑ fold change (PD:control) for each DA tRF in each sample set were plotted. Each group of dots shows the distribution of fold changes across samples for tRFs of the respective category (MT 3′-tRF, Nuc 3′-tRF, etc.). Dots of a given group are shifted to the right of the interval's left boundary according to their p-value: the lower the p-value the higher the shift to the right. As FIG. 2F indicates, each shown interval spans the values 0 through 5 (in -log₁₀). The results were plotted for PFC male comparisons (FIG. 2A), CSF female comparisons (FIG. 2B), CSF male comparisons (FIG. 2C), SER. female comparisons (FIG. 2D), and SER male comparisons (FIG. 2E). Green circles represent PFC samples. Gold circles represent CSF samples. Red circles represent SER samples. As can be seen, many tRFs exhibit considerable fold changes that are also statistically-significant.

FIGS. 3A-3D: tRF characteristics among PFC, CSF, and serum samples. A distribution of the probability that a IRF was expressed in any of X % of the samples in each collection was plotted, Specifically, FIG. 3A and. FIG. 39 show what percentage of identified tRFs (y-axis) is identified in each relative percentage of samples from each collection (x-axis). Green lines show PFC samples (FIG. 3A); gold lines show CSF samples (FIG. 3A-B); and, red lines show SER samples (FIG. 3A-9). FIG. 3C and FIG. 3D describe the identified DA tRFs previously shown in FIG. 2. Specifically, the number of DA tRFs identified for each of the ten tRF types (y-axis) vs. the percentage of the tRFs of each type that were originally identified in the sample group is plotted. Circle areas are sized according to the relative percentage of the total DA tRFs that came from each of the ten tRF types. These results were plotted separately for male (FIG. 3C) and female (FIG. 3D) samples. Green circles represent PFC samples. Gold circles represent CSF samples. Red circles represent SER samples.

FIGS. 4A-4F: tRFs in PFC, CSF, and SER samples that are differentially abundant between CNTRL (Control), PND (Parkinson's Disease without dementia), and PDD (Parkinson's Disease with dementia). t-tests were used to identify the DA tRFs, focusing only on the tRFs in {PFC∪CSF∪SER}—{MINTbase}. These analyses were carried out separately for PFC, CSF, and SER. The Venn diagrams describe the overlap between DA tRFs identified in each context (FIGS. 4A-4E) with those originally identified in FIGS. 1C-1F. Each panel describes the overlap of DA tRFs when splitting samples by patient sex, and by collection (PFC, CSF, or SER). Specifically, sample groups are plotted as follows: PFC male (FIG. 4A), CSF female (FIG. 4B), CSF male (FIG. 4C), SER female (FIG. 4D), and. SER male (FIG. 4E). FIG. 4F shows the complement of DA tRFs in PND vs. PDD comparisons. Specifically, retained and plotted are the intersections of DA tRFs that are not in {MINTbase} and also not part of the set of DA tRFs from CNTRL vs. PD, CNTRL vs. PND, or CNTRL vs. PDD comparisons. Green circles represent PFC samples. Gold circles represent CSF samples. Red circles represent SER samples.

FIGS. 5A-5D: Sample quality filtering based on identified tRF reads. The results of sample quality filtering for CSF (FIGS. 5A and 5C) and. SER (FIGS. 5B and 5D). Specifically, FIGS. 5A and 5B represent the log₂ number of reads passing adapter trimming and quality control (y-axis) vs. the number of reads were mapped to tRFs. FIGS. 5C and 5D show the mean reads mapping to each unique tRF in each sample (y-axis) vs. the log₂ number of reads passing adapter trimming and quality control. Gold circles represent CSF samples, while red circles represent SER samples. Grey rectangles mask samples that contain fewer than 100,000 tRNA space reads and have been excluded from analyses.

FIGS. 6A-6F: tRF profiles by structural type. tRFs derived from either the nuclear or the mitochondrial genome are annotated based on which one of five structural categories they belong. The sum RPM of tRFs belonging to each of the ten possible sets (five structural categories for nuclear and five structural categories for mitochondrial tRFs) as a percentage of the total RPM in each sample are calculated, and the mean and S.E.M. are plotted for PFC (FIG. 6A), CSF (FIG. 6B), and SER (FIG. 6C). FIGS. 6D-6F: the mean and S.E.M on a log₂ scale. S.E.M. is propagated to log₂ per the formula: log₂ es_{x̅}/x̅. RPM for any particular tRF of any of the ten tRF types. Green bars represent PFC PD samples, orange bars represent CSF PD samples, and red bars represent SER PD samples. Grey bars represent controls for each of PFC, CSF, and SER.

FIGS. 7A-7F: IRF profiles by length. In these datasets, tRFs can exist as mature short RNAs with lengths ranging from 16 to 48 nt. The sum RPM of tRFs for all tRFs of a particular length as a percentage of the total RPM in each sample is calculated, and then the mean and S.E.M. for nuclear tRFs only from PFC (FIG. 7A), CSF (FIG. 7B), and SER (FIG. 7C) samples, and for mitochondrial tRFs only for PFC (FIG. 7D), CSF (FIG. 7E), and SER (FIG. 7F) were plotted. Green bars represent PFC PD samples, orange bars represent CSF PD samples, and red bars represent SER PD samples. Grey bars represent controls for each of PFC, CSF, and SER.

### DETAILED DESCRIPTION OF THE INVENTION

Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used. it is also to be understood that the terminology used herein is for the purpose of describing particular embodiments only, and is not intended to be limiting.

As used herein, the articles “a” and “an” are used to refer to one or to more than one .e., to at least one) of the grammatical object of the article. By way of example, “an element” means one element or more than one element.

As used herein when referring to a measurable value such as an amount, a temporal duration, and the like_{;} the term “about” is meant to encompass variations of +20% or within 10%, 9%, 8%, 7%, 6%, 5%, 4%, 3%, 2%, 1%, 0.5%, 0.1%, 0.05%, or 0.01% of the specified value, as such variations are appropriate to perform the disclosed methods. Unless otherwise clear from context, all numerical values provided herein are modified by the term about.

“About” as used herein when referring to a measurable value such as an amount, a temporal duration, and the like, is meant to encompass variations of ±20% or ±10%, more preferably +5%, even more preferably ±1%, and still more preferably ±0.1% from the specified value, as such variations are appropriate to perform the disclosed methods.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a. 50% or greater change in expression levels.

By “complementary sequence” or “complement” is meant a nucleic acid base sequence that can form a double-stranded structure by matching base pairs to another polynucleotide sequence. Base pairing occurs through the formation of hydrogen bonds, which may be Watson-Crick, wobble, Hoogsteen or reversed Hoogsteen hydrogen bonding, between complementary nucleobases. For example, adenine and thymine are complementary nucleobases that pair through the formation of hydrogen bonds. In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the biomarker to be detected.

The phrase “differentially abundant” refers to differences in the quantity and/or the frequency of a biomarker present in a sample taken from subjects having a disease, or condition, as compared to a control subject. A biomarker can be differentially present in terms of quantity, frequency or both. A polypeptide or polynucleotide is differentially present between two samples if the amount or frequency of the polypeptide or polynucleotide in one sample is statistically significantly different (either higher or lower) from the amount of the polypeptide or polynucleotide in the other sample, such as reference or control samples. Alternatively or additionally, a polypeptide or polynucleotide is differentially present between two sets of samples if the amount or frequency of the polypeptide or polynucleotide in samples of the first set, such as diseased subjects' samples, is statistically significantly (either higher or lower) from the amount of the polypeptide or polynucleotide in samples of the second set, such as reference or control samples. A biomarker that is present in one sample, but undetectable in another sample is differentially present.

A “disease” is a state of health of an animal wherein the animal cannot maintain homeostasis, and wherein if the disease is not ameliorated then the animal's health continues to deteriorate. A “disease subtype” is a state of health of an animal wherein animals with the disease manifest different clinical features or symptoms. For example, Alzheimer's disease includes at least three subtypes, inflammatory, non-inflammatory, and cortical.

A “disorder” as used herein, is used interchangeably with “condition,” and refers to a state of health in an animal, wherein the animal is able to maintain homeostasis, but in which the animal's state of health is less favorable than it would be in the absence of the disorder. Left untreated, a disorder does not necessarily cause a further decrease in the animal's state of health.

By “effective amount” is meant the amount required to reduce or improve at least one symptom of a disease relative to an untreated patient. The effective amount of active compound(s) used to practice the present invention for therapeutic treatment of a disease varies depending upon the manner of administration, the age, body weight, and general health of the subject.

As used herein “endogenous” refers to any material from or produced inside an organism, cell, tissue or system.

The term “expression” as used herein is defined as the transcription and/or translation of a particular nucleotide sequence driven by its promoter.

By “fragment” is meant a portion of a polynucleotide or nucleic acid molecule. This portion contains, preferably, at least 10%, 20%, 30%, 40%, 50%, 60%, 70%, 80%, 90%, 91%, 92%, 93%, 94%, 95%, 96%, 97%, 98%, or 99% of the entire length of the reference nucleic acids. A fragment may contain 10, 20, 30, 40, 50, 60, 70, 80, 90, or 100, 200, 300, 400, 500, 600, 700, 800, 900, 1000, 1500, 2000 or 2500 (and any integer value in between) nucleotides. The fragment, as applied to a nucleic acid molecule, refers to a subsequence of a larger nucleic acid. The fragment can be an autonomous and functional molecule. A fragment may contain modifications at neither, one, or both of its termini. A modification can include but is not limited to a phosphate, a cyclic phosphate, a hydroxyl, and an amino acid. A “fragment” of a nucleic acid molecule may be at least about 10 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

“Similar” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are similar at that position. The percent of similarity between two sequences is a function of the number of matching or similar positions shared by the two sequences divided by the number of positions compared X 100. For example, if 6 of 10 of the positions in two sequences are matched or similar then the two sequences are 60% similar. By way of example, the DNA sequences ATTGCC and TATGGC share 50% similarity. Generally, a comparison is made when two sequences are aligned in a way that maximizes their similarity.

“Instructional material,” as that term is used herein, includes a publication, a recording, a diagram, or any other medium of expression that may be used to communicate the usefulness of the compounds of the invention. In some instances, the instructional material may be part of a kit useful for effecting alleviating or treating the various diseases or conditions recited herein. Optionally, or alternately, the instructional material may describe one or more methods of alleviating the diseases or conditions in a cell or a tissue of a mammal. The instructional material of the kit may, for example, be affixed to a container that contains the compounds of the invention or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound. “Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. The term “tRF” stands for tRNA-derived fragment, The term is used to refer to non-coding RNAs that are generated from a tRNA locus and are typically shorter than the locus' corresponding tRNA product. tRFs have typical lengths that range from 10 to 50, and possibly more, nucleotides. For generality, in this discussion, we use the term “tRF” to refer to a fragment that could be derived from the corresponding “precursor tRNA” molecule and either contain a post-transcriptionally added modification (e.g. “CCA”, a “−1” nucleotide as e.g. in the case of tRNAi^{HisGTG}, etc.) or not. As such, the term “tRF” could also refer to the entire “mature tRNA” (typical length of 75 nucleotides), or the entire mature tRNA without the CCA addition (typical length 72 nucleotides).

Generally speaking, the term “mitochondrial tRFs” is used to refer to tRNA fragments whose parental tRNA is located in the mitochondrial genome. Exceptions include: a) several tRNAs of mitochondrial origin that were recently found to be present in the nuclear genome; b) several sequences in the nuclear genome that were recently found to be similar to tRNAs of mitochondrial origin. The term “nuclear tRFs” is used to refer to tRNA fragments whose parental tRNA is located in the nuclear genome.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By “isolated polynucleotide” is meant a nucleic acid molecule (e.g., a DNA or an RNA) that is free of the nucleic acid sequences which flank it in the naturally-occurring genome of the organism from which the nucleic acid molecule of the invention is derived. The term therefore includes, for example, a recombinant DNA that is incorporated into a vector; into an autonomously replicating plasmid or virus; or into the genomic DNA of a prokaryote or eukaryote; or that exists as a separate molecule (for example, a tRF, cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

The term “oligonucleotide panel” or “panel of oligonucleotides” refers to a collection of one or more oligonucleotides that may be used to identify DNA (e.g. genomic segments comprising a specific sequence, DNA sequences bound by particular protein, etc.) or RNA (e.g. mRNAs, microRNAs, tRNAs etc.) or fragments thereof through hybridization of complementary regions between the oligonucleotides and the DNA or RNA. If the sought molecule is RNA, it is commonly converted to DNA through a reverse transcription step). The oligonucleotides may include complementary sequences to known DNA or known RNA sequences, The oligonucleotides may be engineered to be between about 5 nucleotides to about 40 nucleotides, or about 5 nucleotides to about 30 nucleotides, or about 5 nucleotides to about 20 nucleotides, or about 5 nucleotides to about 15 nucleotides in length. The term “oligonucleotide panel” or “panel of oligonucleotides” could also refer to a system and accompanying collection of reagents that, in addition to being able to hybridize to molecules containing a complementary sequence, can also ensure that the identified molecule's 3′ terminus matches precisely the 3′ terminus of the sought molecule, or that the identified molecule's 5′ terminus matches precisely the 5′ terminus of the sought molecule, or both: this ability is unlike what can be achieved by conventional assays such as e.g. Affymetrix chips and methods (e.g. “dumbbell-PCR”) and systems (e.g. the Fireplex system of Firefly BioWorks) that can achieve this are now beginning to be available.

The term “operably linked” refers to functional linkage between a regulatory sequence and a heterologous nucleic acid sequence resulting in expression or changes in abundance of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence.

The terms “patient,” “subject,” “individual,” and the like are used interchangeably herein, and refer to a human or non-human mammal, or cells thereof whether in vitro or in situ, amenable to the methods described herein. Non-human mammals include, for example, livestock and pets, such as ovine, bovine, porcine, canine, feline and murine mammals. The term “subject” is intended to include living organisms in which an immune response can be elicited (e.g., mammals). Examples of subjects include humans, dogs, cats, mice, rats, and transgenic species thereof. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which may be hydrolyzed into the constituent monomeric “nucleotides.” The monomeric nucleotides may be hydrolyzed into nucleosides. As used herein polynucleotides include, but are not limited to, all nucleic acid sequences that are obtained by any means available in the art, including, without limitation, recombinant means, i.e., the cloning of nucleic acid sequences from a recombinant library or a cell genome, using ordinary cloning technology and PCR™, and the like, and by synthetic means. The following abbreviations for the commonly occurring nucleic acid bases are used. “A” refers to adenosine, “C” refers to cytosine, “G” refers to guanosine, “T” refers to thymidine, and “U” refers to uridine. The term “RNA” as used herein is defined as ribonucleic acid. The term “recombinant DNA” as used herein is defined as DNA produced by joining pieces of DNA from different sources.

As used herein, the terms “prevent,” “preventing,” “prevention,” and the like refer to reducing the probability of developing a disease or condition in a subject, who does not have, but is at risk of or susceptible to developing a disease or condition.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The terms “purified,” or “biologically pure” refer to material that is free to varying degrees from components which normally accompany it as found in its native state. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. By “reduces” or “decreases” is meant a negative alteration of at least 10%, 25%, 50%, 75%, or 100%.

By “reference” is meant a standard or control. A “reference” is also a defined standard or control used as a basis for comparison.

As used herein, “relative abundance” refers to the ratio of the quantities of two or more molecules of interest (e.g. tRFs) present in a sample. The relative abundance of two or more molecules of interest in a given sample may differ from the relative abundance of the same two or more molecules in a second sample.

As used herein, “sample” or “biological sample” refers to anything, which may contain the biomarker (e.g., polypeptide, polynucleotide, or fragment thereof) for which a biomarker assay is desired. The sample may be a biological sample, such as a biological fluid or a biological tissue or an isolated cell or a collection of isolated cells or an isolated extracellular vesicle or collection of extracellular vesicles. In one embodiment, a biological sample is a tissue from prefrontal cortex. Examples of biological fluids include cerebrospinal fluid, urine, blood, plasma, serum, saliva, semen, stool, sputum, cerebral spinal fluid, tears, mucus, amniotic fluid or the like.

As used herein, the term “sensitivity” is indicates a percentage of biomarker-detected subjects with a particular disease. For example, in a specific assay to detect a percentage of disease-affected subjects in a particular population, the higher detectability of percentage of biomarkers (such as, for example tRFs) related to the diseases being detected corresponds to the higher sensitivity index of that assay.

The terms “short RNA profile” or “RNA profile” or “IRF profile” or “IRF profile” or “tRNA profile” or “tRNA fragment profile” are used interchangeably and refer to a genetic makeup of RNA molecules that are present in a sample, such as a cell, tissue, or subject. Optionally, the abundance of an RNA molecule that is part of an RNA profile may also be sought. Optionally, other attributes of an RNA molecule that is part of an RNA profile may also be sought and include but are not limited to a molecule's location within the genomic locus of origin, the molecule's starting point, the molecule's ending point, the molecule's length, the identity of the molecule's terminal modifications. etc.

The term “signature” or “tRF signature” as used herein refers to a subset of an RNA profile and comprises the identity of one or more molecules that are selected from an RNA profile and optionally one or more of the attributes of the one or more molecules that are selected from the RNA profile.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

The term “therapeutic” as used herein means a treatment and/or prophylaxis. A therapeutic effect is obtained by suppression, remission, or eradication of a disease state.

As used herein, the terms “treat,” treating,” “treatment,” and the like refer to reducing or improving a disease or condition and/or symptom associated therewith. It will be appreciated that, although not precluded, treating a disease or condition does not require that the disease, condition or symptoms associated therewith be completely ameliorated or eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Description

The present invention includes methods and compositions of analyzing tRFs. Described herein are tRFs that are important for disease diagnosis. The present invention utilizes tRF-profiling to identify subjects in need of therapeutic intervention. One or more of the tRFs that are important for disease diagnosis can also serve as therapeutic targets themselves.

In one aspect, the invention provides a method of identifying a subject in need of therapeutic intervention to treat a disease, disease progression, or disease recurrence. The method comprises isolating tRFs from a sample obtained from the subject; characterizing the fragments of tRFs and their relative abundance in the sample to identify a signature, Wherein when the signature is indicative of a diagnosis of the disease of a disease subtype or of a disease recurrence), a treatment of the subject is recommended or provided. It is generally the case that the mechanistic events that are promoted by the tRFs used to determine whether treatment is recommended or provided can be elucidated through additional work. Consequently, one or more of these tRFs can serve as novel therapeutic targets as well.

### tRF Fragments

Analysis of tRF profiles or signatures in one or more cells lead to the discovery of tRF signatures present in healthy cells or diseased cells. tRF signatures of one or more cells, or a tissue may be used to identify a diseased cell, disease progression, or disease recurrence in a subject. Thus, the subject may be identified as in need of therapeutic intervention to delay the onset of, reduce, improve, and/or treat a disease or condition, such as cancer, in a subject in need thereof. One or more of the tRFs that comprise these signatures can also serve as targets of novel therapeutic interventions.

In another aspect, the invention includes a method for identifying tRFs from sequenced reads, typically obtained through next generation sequencing approaches. Various sequencing methodologies and platforms are known in the art. The choice of a platform may be based on the user's and experiment's requirements. In some embodiments, the sequencing method is a high throughput next-generation method. Non-limiting examples of massively parallel signature sequencing platforms are Illumina sequencing by synthesis (Illumina, san Diego Calif.), 454 pyrosequencing (Roche Diagnostics, Indianapolis Ind.), SOLiD sequencing (Life Technologies, Carlsbad, Calif.), Ion Torrent semiconductor sequencing (Life Technologies, Carlsbad, Calif.), Heliscope single molecule sequencing (Helicon Biosciences, Cambridge, Mass.), and Single molecule real time (SMRT) sequencing (Pacific Biosciences, Menlo Park, Calif.).

In certain embodiments the invention comprises analyzing five different and previously-defined structural categories of tRFs that overlap the mature tRNA. Further, these analyses may distinguish among these tRFs based on whether they originate in nuclearly- or mitochondrially-encoded tRNAs. Thus, the analyses can comprise as many as 10 different groups: 3′-tRFs, 5′-tRHs, and i-tRFs from the mitochondrion (MT) and the nucleus (Nuc), respectively.

In certain embodiments the method for characterizing the tRNA fragments in this invention comprises at least one assessment selected from the group consisting of sequencing the tRFs, measuring overall abundance of one of the tRFs, measuring a relative abundance of the one tRF to a reference, assessing a length of the one tRF, identifying starting and ending points of the one tRF, identifying genomic origin of the one tRF, and identifying a terminal modification of the one tRF,

Diagnostics

Samples from subjects suffering from a disease or a condition have a specific tRF-profile in the cell or cells that are diseased, including cells from the subject suffering from the Parkinson's Disease (PD). One or more of these tRFs could enter circulation and be found in, for example, extracellular vesicles. In such an event, the profile of tRFs contained in isolated exosomes could prove equally powerful in helping identify the disease or condition as the cells of the ailing tissue. Moreover, the disease or condition could also affect the tRF profile of cells in other adjacent or distant tissues. In such an event, the tRF profile of cells from these adjacent or distant tissues could prove equally powerful in helping identity the disease or condition as the cells of the ailing tissue. It is noted that the disease or a condition can affect the composition and abundance of tRNAs, of tRFs, or both.

In one aspect, a method of diagnosing, identifying or monitoring a disease in the subject comprises isolating tRFs from a cell obtained from the subject; analyzing levels of the tRFs present in the cell; wherein a differential in the level of measured tRF's as compared to a reference is indicative of a diagnosis or identification of Parkinson's disease in the subject; and providing a treatment regimen to the subject dependent on the differential in the level of measured tRFs as compared to the reference.

Identifying the existence of a disease or condition by identifying a tRF profile associated with the disease or condition in a sample obtained from the subject allows recommending or offering the subject a personalized treatment. In one aspect, the invention includes a method of identifying a cell's tRF population to establish and treat the onset of a disease, or the state of the disease, or that disease has recurred in a subject in need thereof comprising isolating fragments of tRFs from a sample (e.g. a cell) obtained from the subject; characterizing the fragments of tRF, which can include assessing one or more of, overall abundance, relative abundance, length of the fragment, starting and ending points of the fragment, terminal modifications, etc., in the sample to identify a. signature, wherein the signature is indicative of the presence of the disease, of the state of the disease, or of disease recurrence; and providing a treatment regimen to the subject dependent on the sampled cell's profile of tRFs.

In certain embodiments, characterizing the tRFs and their relative abundance can identify subjects in need of treatment. In certain embodiments the tRFs and their relative abundance vary between the normal state subject as compared to a subject at risk of or suffering from PD. In certain embodiments, the tRFs and their relative abundance compared to control subjects differ between subjects suffering from PD with dementia and subjects suffering from PD without dementia. In certain embodiments, the tRFs and their relative abundance vary with the gender of the subject. In certain embodiments, the tRFs and their relative abundance vary with the body fluid, cell or tissue sample.

In certain embodiments, the relative abundance of the tRFs that are present in the RNA profile can identify subjects in need of treatment. In another approach, diagnostic methods are used to assess tRF profiles in a biological sample relative to a reference (e.g., tRF profile in a healthy cell or tissue or body fluid in a corresponding control sample). Examples of a body fluid and tissues may include, but are not limited to amniotic fluid, bile, blood serum, plasma, cerebrospinal fluid, plasma and prefrontal cortex.

In certain embodiments, the sample, such as a cell or tissue or body fluid is obtained from the subject. In another embodiment, the cell or tissue or body fluid is isolated from the sample. In another embodiment, the cell or tissue is isolated from a body fluid.

In certain embodiments, a signature of tRFs or a presence or absence of specific tRFs are indicative a diagnosis of a disease or condition. In some embodiments, the signature of tRFs distinguishes a normal state as compared to a disease state or a condition.

In some embodiments, the signature of tRF comprises at least one sequence selected from the group consisting of: SEQ ID NOs: 53135-63850. In some embodiments, the signature comprises at least one sequence among those listed in SEQ ID NOs; 1-53134 that are not contained in SEQ ID NOs: 53135-69512.

In some embodiments, the methods or assays described herein comprise analyzing the presence or absence or the signature of tRFs in a disease or condition, disease recurrence, or disease progression selected from the group consisting of a cancer, a brain disease, a glaucoma, or a genetically predisposed disease or condition.

In general, characterizing the tRFs of this invention identifies a signature that may be indicative of a diagnosis of a disease or condition, the onset of a disease or condition, or the recurrence of a disease or condition. Moreover, characterizing the tRFs of this invention identifies one or more molecules that can serve as therapeutic targets themselves.

The character of the tRFs in the sample may be compared with a reference, such as other tRFs present within the cell, a healthy cell or a diseased cell will yield a relative abundance of the tRFs to identify a signature. Alternatively, the abundance of two or more tRFs, two or more tRNAs, or at least one tRF and at least one tRNA may be compared to identify a signature. Alternatively, the signature may be established by comparing the locations of the two or more tRFs within the genomic loci of origin, the starting and ending points of the fragments, the lengths of the fragments, and any other feature of the fragments as compared to other tRFs within the same sample or another sample or reference to distinguish a diseased state or condition, a propensity to develop a disease or condition, and/or the absence of a disease or condition. The skilled artisan will appreciate that the diagnostic can be adjusted to increase the sensitivity or specificity of the assay. In general, any significant increase (e.g., at least about 10%, 15%, 30%, 50%, 60%, 75%, 80%, or 90%) in the level of a polynucleotide or polypeptide biomarker in the subject sample relative to a reference may be used to diagnose a diseased state, a. propensity to develop a disease or condition, and/or the absence of a disease or condition.

Accordingly, a tRF profile may be obtained from a sample from a subject and compared to a reference tRF or tRNA fragment profile obtained from a reference cell or tissue or body fluid, so that it is possible to classify the subject as belonging to or not belonging to the reference population. The association may take into account the presence or absence of one or more tRF or tRNA fragments in a test sample and the frequency of detection of the tRF or tRNA fragments in a test sample compared to a control. The association may take into account both of such factors to facilitate a diagnosis of a disease or condition. The association may take into account both of such factors to guide recommending or offering a treatment, In one embodiment, the reference is the identity and abundance level of the tRF or tRNA fragments present in a control sample, such as non-diseased cell, a cell obtained from a patient that does not have the disease or condition at issue or a propensity to develop such a disease or condition. In another embodiment, the reference is a baseline level of the tRF or tRNA fragment presence and abundance in a biologic sample derived from the patient prior to, during, or after treatment for the disease or condition. In yet another embodiment, the reference is a standardized curve.

### Methods of Use

In one aspect, the invention includes a method of identifying a subject in need of treatment. The method comprises isolating fragments of tR.Fs from a sample obtained from the subject; characterizing the identity of the tRFs and their relative abundance in the sample to identify a signature, wherein the signature differs depending on the sex of the subject; and recommending or providing a personalized treatment regimen or a disease prognosis to the subject. In some embodiments, the signature comprises at least one sequence selected from the group consisting of SEQ ID NOs: 53135-63850. In some embodiments, the signature comprises at least one sequence among those listed in SEQ ID NOs: 1-53134 that are not contained in 53135-69152.

In another aspect, the method described herein includes diagnosing, identifying or monitoring the onset or recurrence of a disease or condition, such as a PD, in a subject in need of therapeutic intervention. In one embodiment, the method includes isolating RNA from a cell, tissue or body fluid obtained from the subject; hybridizing the RNA to a panel of oligonucleotides engineered to detect tRFs; analyzing an identity and levels of the tRFs present in the cell; wherein a differential in the identity or measured tRFs' levels to the reference is indicative of a diagnosis or identification of a disease or condition, such as PD, in the subject; and providing a treatment regimen to the subject dependent on the differential in the identity and measured tRFs' levels to the reference.

The non-coding RNAs of interest (tRFs or tRNAs) may be isolated by a method known in the art or selected from the group consisting of size selection, amplification and sequencing.

In some embodiments, the size of the tRFs is in the range of about 10 nucleotides to about 70 nucleotides are isolated. The range of sizes may include, but are not limited to, from about 15 nucleotides to about 50 nucleotides, and from about 20 nucleotides to about 45 nucleotides. The size of the tRFs fragments may be about 10 nucleotides, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or about 70 nucleotides.

In one embodiment, the signature is obtained by hybridization to a single oligonucleotide, or to a panel of oligonucleotides, such as those that comprise at least two or more oligonucleotides that selectively hybridize to the tRFs. To prepare the sample for characterization, the tRFs may be amplified prior to the hybridization.

### Monitoring

Methods of monitoring subjects that are at risk of developing a disease or condition, or are at risk of disease or condition recurrence, or who are receiving therapeutic intervention to reduce, improve, or treat a symptom of the disease or condition, such as breast cancer, are also useful in determining whether to administer treatment and in managing treatment. Provided are methods where the tRFs are measured and characterized. In some cases, the tRFs are measured and characterized as part of a routine course of action. In other cases, the tRFs are measured and characterized before and again after subject management or treatment. In these cases, the methods are used to monitor the onset of a disease or condition, the recurrence of the disease or condition, the status of the disease or condition, or a propensity to develop such disease or condition, e.g., Parkinson's disease.

For example, characterization of tRFs or signatures can be used to monitor a subject's response to certain treatments. Such characterization can be used to monitor for the presence or absence of the disease or condition. The changes in the relative abundance or tRF signature delineated herein before treatment, during treatment, or following the conclusion of a treatment regimen may be indicative of the course of the disease or condition, progression of disease or condition, or response to treatment. In some embodiments, characterization of tRFs or signatures may be assessed at one or more time points (e.g., 2, 3, 4, 5). Analysis of the tRFs is made, for example, using a size selection, amplification, and sequencing, or other standard method to determine the tRF profile. If desired, the tRF profile is compared to a reference to determine if any alteration in the tRF profile is present. Such monitoring may be useful, for example, in assessing the efficacy of a particular treatment in a patient. Therapeutics that normalize the tRF profile are taken as particularly useful.

### Kits

Kits for diagnosing, identifying or monitoring a disease or condition, such as PD with or without dementia, are included. In one aspect, the invention includes a. panel of engineered oligonucleotides comprising a mixture of oligonucleotides that are about 5 to about 15 nucleotides (nts) in length and capable of hybridizing RNAs (e.g. tRFs, full-length tRNAs, etc.), wherein the RNAs and RNA fragments are less than about 70 nts in length. In another aspect, the invention includes a kit for high-throughput analysis of RNAs or RNA fragments in a sample comprising the panel of engineered oligonucleotides of this invention along with hybridization reagents and RNA isolation reagents. In another aspect, the invention includes a kit for high-throughput analysis of RNAs or RNA fragments in a sample comprising a set of specially designed TaqMan® assays aimed at measuring the abundance of molecules mentioned in this invention. Alternatively, the kit could include: a specially designed TaqMan® Gene Expression Assays, TaqMan® Low Density Array-micro fluidic cards: a set of end-point specific assays such as dumbbell-PCR; a set of miR-ID assays. Other kits with variations on the components and oligonucleotide panels may be used in the context of the present invention. For example, the panel of engineered oligonucleotides or specially-designed kit may be specific to a cell type, disease type, stage of disease, or other aspect that may differentiate RNA fragment signatures. The kits and oligonucleotide panel may also be used to identify agents that modulate disease, or progression of disease in in vitro or in vivo animal models for the disease. In some embodiments, the subject in need thereof is a human.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure, The Results of the experiments disclosed herein are now described.

### Materials and Methods

### Datasets

Short RNA-sequencing (RNA-seq) data were obtained from two independent collections. Prefrontal cortex (PFC) data (Brodmann's area nine) were obtained from NM GEO: the 29 PD samples are from series #GSE72962, (Hoss et al, Front Aging Neurosci 2016; 8: 36.) whereas the 33 control samples are from series #GSE64977 (Hoss et al., BMC Med Genomics 2015; 8: 10). The 67 PD CSF samples, 69 CSF controls, 61 PD SER samples, and 71 SER controls were obtained from dbGap, reference #phs000727. (Burgos et al., PLoS One 2014; 9(5): e94839)

### Preprocessing of Sequenced Reads and tRF Profiling

First, short RNA-seq samples were quality-trimmed and adapters removed using the cutadapt (v1.5) tool. The MINTmap pipeline was used to profile and quantify tRFs in these samples. MINTmap only uses exact matches and distinguishes between exclusive tRFs that can only be found inside “tRNA space” and ambiguous tRFs that exist both inside and outside of tRNA space, Next, the Threshold-seq algorithm was used to calculate an adaptive threshold for each short RNA-seq sample. All tRFs that exceeded this threshold entered the downstream analysis. Finally, for all tRFs in each sample that exceeded the Threshold-seq threshold, a normalized value of abundance in reads per million (RPM) was calculated.

### Sample Quality Control

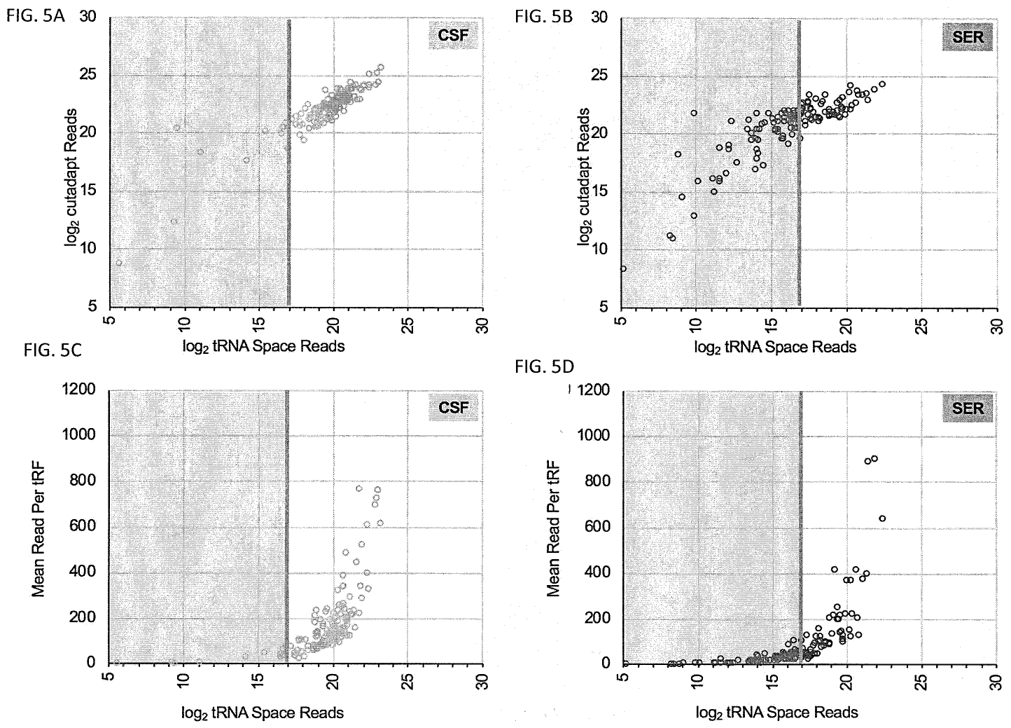

Extracting RNA from biofluids can be challenging due to instability of miRNA profiles derived from CSF and SER samples. In these analyses, a positive correlation between the number of reads after cutadapt quality-trimming of the CSF and SER collections and the number of reads that are identified as tRFs was observed (FIGS. 5A-5B). The correlation appears to break down in those samples where fewer reads survive quality-filtering. Looking at the average numbers of reads that map to the tRNA space allowed to determine the minimum number of trimmed reads needed to identify all unique tRFs: this appears to be achieved in samples where ≥100,000 reads map to tRFs (FIG. 5C-5D). Importantly, this minimum required number of reads mapping to tRNA space allowed to confidently identify samples in which tRFs are either present at very low levels, or not expressed at all. 127 CSF samples and 65 SER samples satisfied the requirement that ≥100,000 reads map to tRNA space.

### Sequence Batch Correction

The PFC samples that were analyzed were originally sequenced in three batches (SEQ ID NOs: 1-53134). To correct for batch effects, the RPM expression data was processed using the ComBat algorithm from Bioconductor (release 3.6). Combat-processed reads were used for all downstream analysis.

### Statistical Analyses

All analyses were run R version 3.3.0. Two-tailed t tests were used to identify differentially-abundant tRFs, retaining those which showed a significant difference in mean across sample categories (p-value≤0.05). To construct classifier models, the rpart package was used for regression trees, and the DiscriMiner package for Partial Least Squares—Discriminant Analysis (PLSDA). These two methods were chosen for their opposing qualities: RPART regression trees seek to define a step-by-step classification method that separates defined sample groups using “yes”/“no” decisions on quantitative data; PLSDA transforms collective observations about samples so that they most readily discriminate defined sample groups.

Monte Carlo simulations were used to evaluate the performance of these models. For each sample group, the datasets were randomly split into a training set comprised of 60% of the control and 60% of the disease samples, and a test set comprising the remaining 40% of control and disease samples. Using the training set, a classifier was built and used to determine the status of each sample in the test set. To adequately measure model performance, this was iterated over random sampling a total of N² times, where N is the sum total of the disease and control samples. To compute sensitivity and specificity, the total number of true positive, true negative, false positive, and false negative calls were tallied and made in each of the N² iterations.

Datasets corresponding to samples from prefrontal cortex (PFC), cerebrospinal fluid (CSF), and serum(SER) were obtained from NIH's GEO and dbGap. The datasets were subjected to the quality filtering described elsewhere, herein. After filtering, 62 PFC, 127 CSF, and 65 SER samples were further analyzed. All of the PFC samples were obtained from male patients. The clinical characteristics of these patients are listed in Table 1.

TABLE 1

| CLINICAL | | | |
|---|---|---|---|
| CHARACTERISTICS | PFC | CSF | SER |
| Control Samples | 33 | 64 | 31 |
| PD Samples | 29 | 63 | 34 |
| PD with Dementia | 11 | 24 | 12 |
| PD without Dementia | 18 | 39 | 22 |
| Male - Control | 33 | 37 | 15 |
| Male - PD | 29 | 37 | 23 |
| Female - Control | 0 | 27 | 16 |
| Female - PD | 0 | 26 | 11 |
| Age at Death | 72.5 ± 13.22 | 79.3 ± 8.6 | 80.9 ± 8.0 |
| (Mean ± SD) | | | |
| Motor Onset | 66.5 ± 9.8 | N/A | N/A |
| (Mean ± SD) | | | |

### Example 1

### The tRF Abundance Profiles Differ Among PFC, CSF and SER

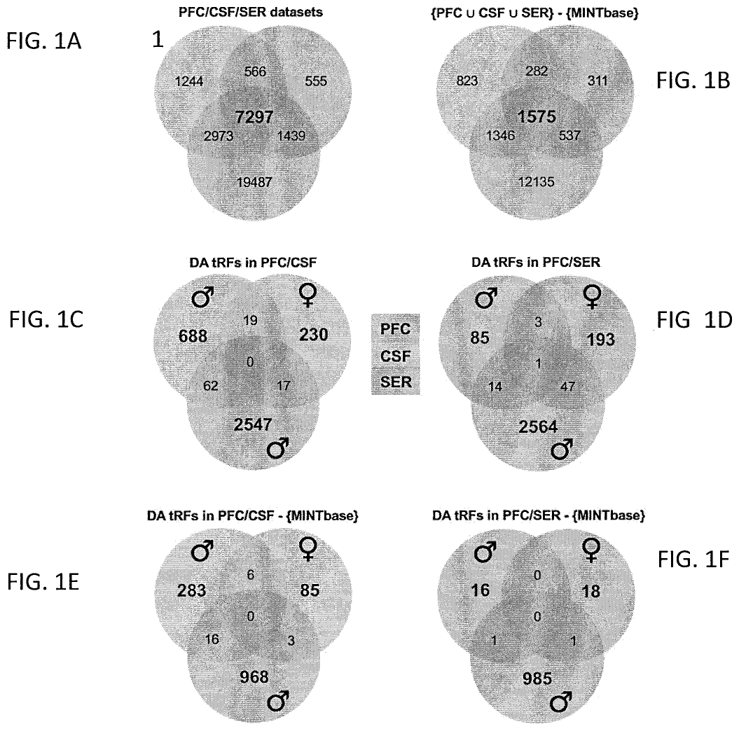

By applying MINTmap and Threshold-seq on the datasets that survived the filtering, 31,196 tRFs in the PFC collection, 12,608 tRFs in the CSF collection, and 9,857 tRFs in the SER collection were identified. The corresponding tRF sequences are listed in SEQ ID NOs: 1-53134. Some tRFs appeared in two or more datasets. In total, there were 33,561 unique tRFs discovered across the three collections. In this work, “{PFC∪CSF∪SER}” is used to denote this set of unique tRFs. FIG. 1A shows how many tRFs are shared among the three collections. 58% of the tRFs in {PK∪CSF∪SER} are unique to the PFC samples.

As mentioned elsewhere herein, MINTmap distinguishes between tRFs that can arise exclusively from annotated tRNAs (“exclusive” tRFs) and “ambiguous” tRFs. The sequences of the latter exist in tRNA space as well as elsewhere on the genome. Of all PFC tRFs, 22,477 (72.05%) are exclusive. Similarly, 9,390 (74.47%) of all CSF tRFs are exclusive, as are 7,609 (77.19%) of all SER tRFs.

### Example 2

### All Five Structural Categories are Represented Among the Identified tRFs

The analyses herein focus on the five categories of tRFs that overlap the mature tRNA. The analyses distinguish among these tRFs based on whether they originate in nuclearly- or mitochondrially-encoded tRNAs. Thus, a total of 10 groups 3′-tRHs, 5′-tRFs, 5′-tRHs, and i-tRFs from the mitochondrion (MT) and the nucleus (Nuc), respectively, are considered.

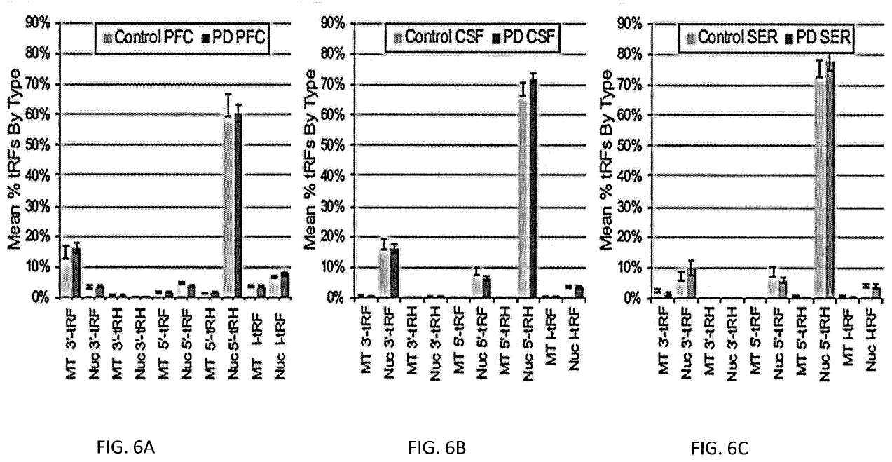

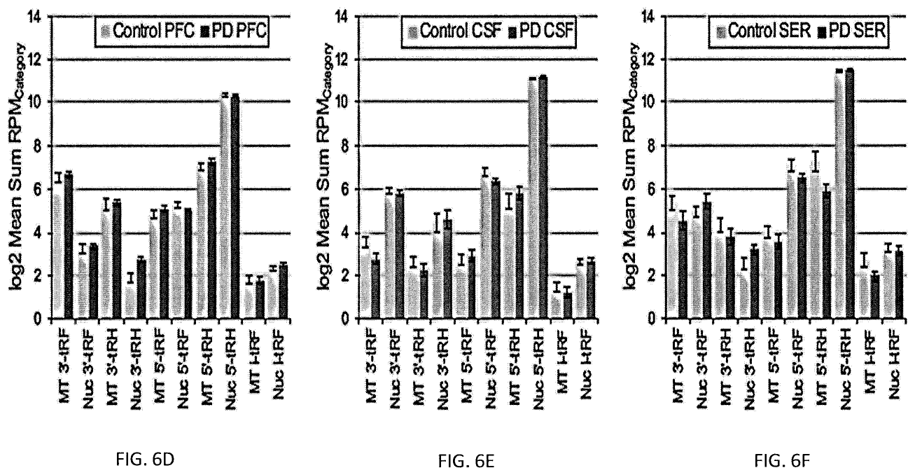

First, relative abundances at the level of tRF categories are examined. FIGS. 6A-6C show that nuclear 5′-tRHs are most abundant in each of the three collections. In PFC, the majority of the remaining reads map to MT 3′-tRFs and nuclear i-tRFs. On the other hand, in both CSF and SER, most of the remaining reads map to nuclear 5′-tRFs, i-tRFs and 3′-tRFs.

However, this representation is skewed in that it does not account for the fact that different numbers of distinct tRFs are present in each tRF category. To address this bias, the relative abundances at the level of individual tRFs was examined separately for each of the 10 groups under consideration. Specifically, for each sample in a collection, e.g. PFC, the total RPM supporting a tRF category was divided, e.g. nuclear 5′ ARFs, by the number of distinct tRFs in the category. This number was then averaged across all the samples within the same collection. This calculation for the CSF and SER. collections was repeated. The results are summarized in FIGS. 6D-6F. This representation demonstrated that the average abundance per tRF is considerably less polarized across the 10 considered tRF groups than FIG. 6A-6C would suggest. For example, in the CSF collection, the nuclear 3′-tRFs account for a larger percentage of total reads than do 5′-CRFs. However, the reads land on comparatively fewer 5′-tRFs, which essentially puts the individual tRFs in these categories on equal footing in terms of relative abundances.

### Example 3

### The Observed tRFs have Length Distributions that are Sample-Source-Dependent

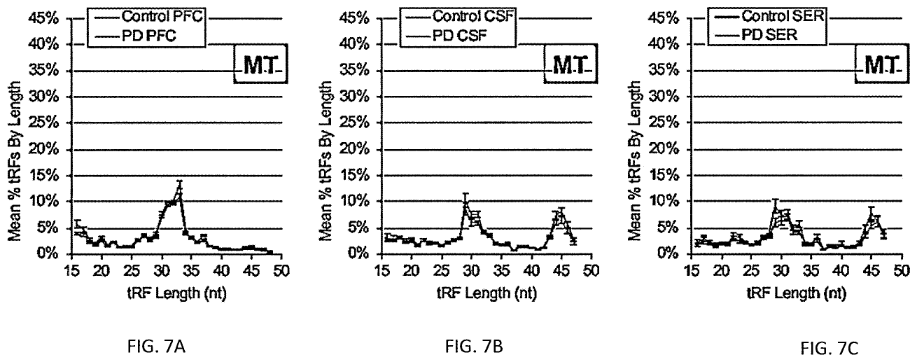

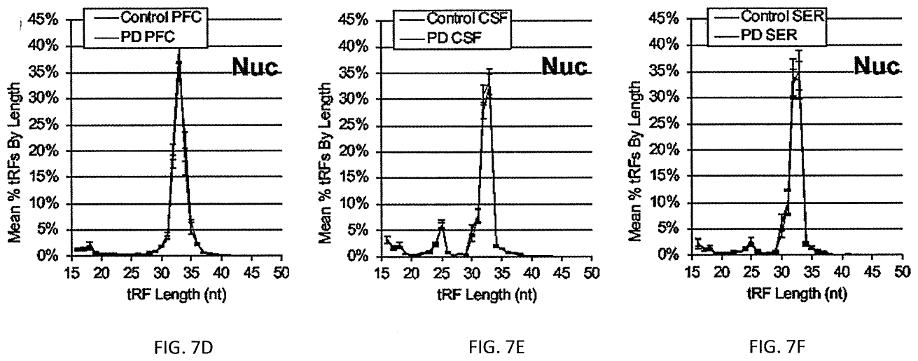

The average abundance of tRFs of a specific length separately in each of the three collections was examined. The distribution of RPM sums as a function of length, where all lengths are between 16 and 48 nt was considered. Overall, the three collections do not show any appreciable length differences between control (CNTRL) and PD. This is true for both MT tRFs (FIG. 7A-7C) and nuclear tRFs (FIG. 7D-7F) from all three collections. Even though no intra-collection differences were observed, it was found that there are inter-collection length differences. In particular, the majority of nuclear tRFs in the PFC collection are of lengths 32-34 nt (FIGS. 7D-7F). On the other hand, PFC MT tRFs are primarily 31-33 nt long (FIG. 7A), whereas in both CSF and SER the MT tRFs have peaks at both 29-31 nt and 44-46 nt (FIG. 7B-7C).

### Example 4

### Many of the Discovered tRFs are Unique to a Given Cellular Context

As mentioned above, numerous tRFs have been reported by now in different cellular contexts. This is an important consideration for attempts to develop potential biomarkers. tRFs that appear in multiple cellular contexts do not have the desirable tissue- and disease-state specificity that is expected of a good biomarker, and thus should be excluded from consideration.

With that in mind, MINThase, which is currently the largest repository of tRFs from human samples, was used. In particular, release 2.0 of MINTbase contains 26,744 distinct tRFs mined from 11,721 datasets and a multitude of contexts, In what follows, this set of 26,744 tRFs is denoted as “{MINTbase}.” MINTbase contains tRFs from 32 cancer types from TCGA, 452 samples from the 1000 Genomes Project for which RNA-seq data were made available, as well as several hundred more samples from other studies, both non-cancer and cancer. Release 2.0 of MINTbase also contains 7,405 distinct tRFs that were mined from the 535 brain tissue samples of TCGA's project on lower grade glioma (LGG). Any and all {MINTbase} tRFs that are present among the 33,561 tRFs of {PFC∪CSF∪SER} that is described above were removed as their presence in {MINTbase} indicates that their expression is neither PD-specific nor tissue-specific. The tRF set {PFC∪CSF∪SER}−{MINTbase} that includes 17,009 tRFs was created. Specifically, {PFC∪CSF∪SER}−{MINTbase} retained only 15,593 (49.98%) of the original PFC tRFs, 4,026 (33.33%) of the original CSF tRFs, and 2,705 (27.44%) of the original SER tRFs (FIG. 1B). The {PFC∪CSF∪SER}−{MINTbase} tRFs are present in the samples from PD patients and the matching CNTRL, and absent from the 11,271 datasets of {MINTbase}. In other words, they are as tissue- and as disease-context specific as allowed by the currently available data on tRFs. FIG. 1B shows how the tRFs that remain in {PFC∪CSF∪SER}−{MINTbase} are distributed across the three collections (PFC, CSF, SER).

### Example 5

### Multiple tRFs are Differentially Abundant Between CNTRL and PD in All Three Collections

The tRFs that are differentially abundant (DA) between health and the PD state were sought. Specifically, the t statistic and p-value of comparison of each individual tRF in CNTRL and PD groups of batch-corrected PFC data was computed, and a t test was performed of each individual tRF in CNTRL and PD groups in CSF and SER. data with a p-value threshold set to 0.05. Because it is known that a person's sex modulates tRF expression, DA tRFs were sought separately in samples from male and female donors.

For the full collection of tRFs in the {PFC∪CSF∪SER} set, the numbers of identified DA tRFs and their overlaps are summarized in FIGS. 1C-1D, for males and females. FIGS. 1E-1F show how the overlaps change when tRFs that are in the {MINTbase} set are removed from consideration. Note how 19 tRFs are DA between PD and CNTRL CSF in both males and females: of these, six have not been seen yet in other contexts (i.e., these six are not in part of the {MINTbase} collection of tRFs). By contrast, only three tRFs are DA between PD and CNTRL SER in both males and females: however, these three tRFs are in {MIN base} and thus are considered to not be specific to the PD context.

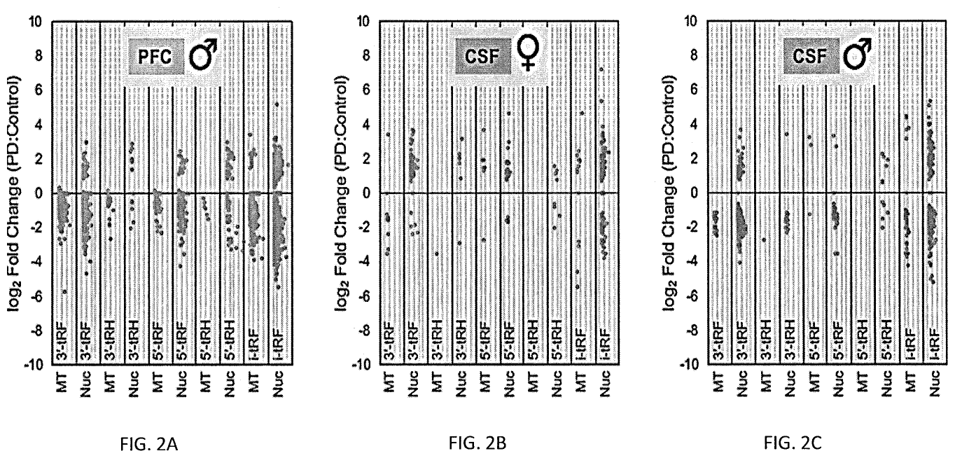

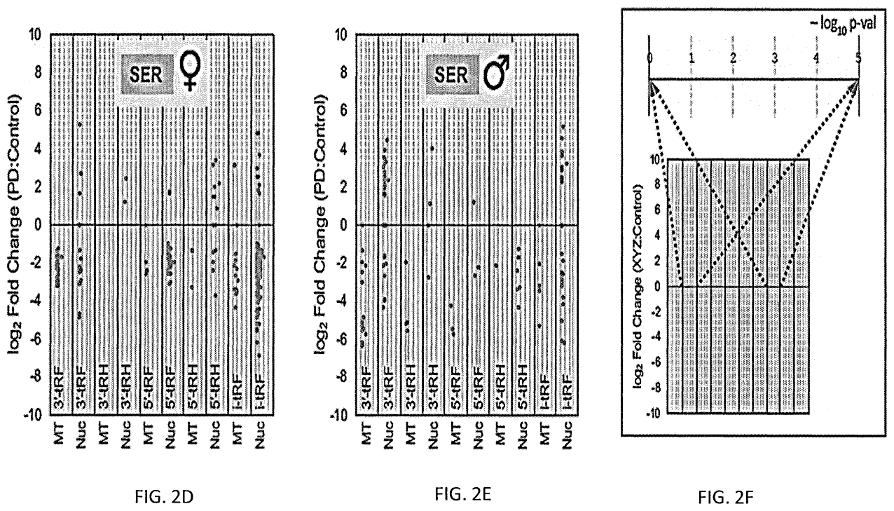

tRFs that are DA in the original comparison of PFC brain tissue (all male donors) overlap with male comparisons in biofluids. 62 tRFs are DA between PD and CNTRL samples in both CSF and PFC: 16 of the 62 are novel and have not been seen in other contexts. FIG. 2 shows the logₑ fold change between PD and CNTRL (i.e. the log₂ of the ratio of mean PD over mean CNTRL for each tRF) for all DA tRFs and across all comparisons. The data are provided separately for each of the five tRF categories and two genomic origins (nucleus and MI). In each plot, individual points represent DA tRFs, with the point's Y-value capturing the magnitude of the tRF's observed fold change (log₂) and the X-value capturing the −log₁₀ of the tRF's p-value (the further right, the lower the p-value).

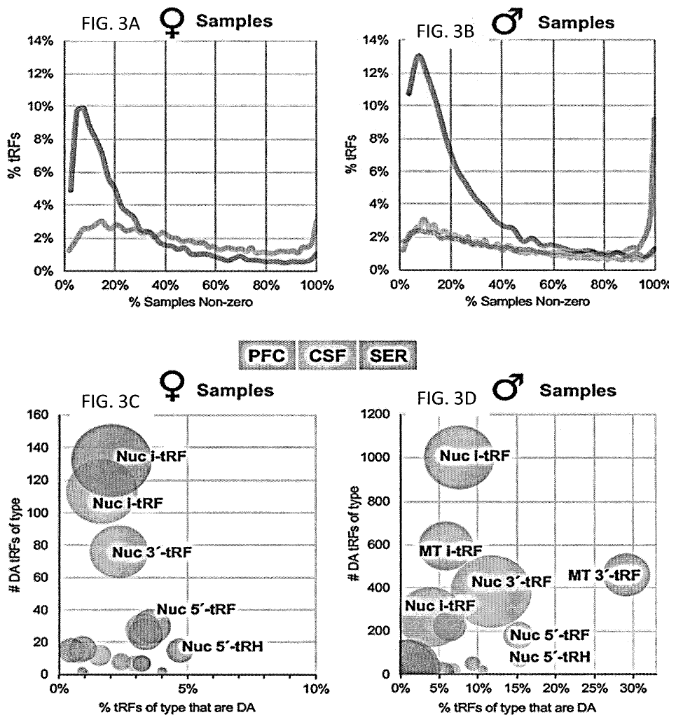

Next, the distribution of tRFs across the collections was examined (FIG. 3A-B). As can be seen, there is a characteristic difference among the three collections. In PFC, ˜10% of tRFs are present in nearly all samples. On the other hand, in SER, most tRFs are present only in small groups of samples. In CSF, multiple tRFs are present in multiple samples. In other words, there exists a large number of tRFs from which DA tRF biomarkers could be drawn.

Lastly, examination was done to evaluate which of the 10 groups (five structural categories x 2 genomic origins) contribute to the DA tRFs of the {PKC∪CSF∪SER} set (FIGS. 3C-3D). Specifically, the number of DA tRFs in a given group (Y-axis) was plotted as a function of the percentage of tRFs from the same structural group that the DA tRFs represent (X-axis). In this FIG, the area of each bubble (and not the bubble's radius) associated with each tRF category is proportional to the number of DA tRFs (from the category), The rendering helps capture the relative relevance of the various tRF types in disease states. For example, in all three collections (PFC, CSF, and SER) it was found that a large number of nuclear i-tRFs that are DA (FIGS. 3C-3D). However, these i-tRFs are a relatively small portion of all i-tRFs that are present. On the other hand, almost one third of the MT 3′-tRFs that are present in PFC are DA between PD and CNTRL. Analogous observations can be made for the other tRF groups,

### Example 6

### Multiple tRFs are Differentially Abundant in the Context of Dementia Associated with PD

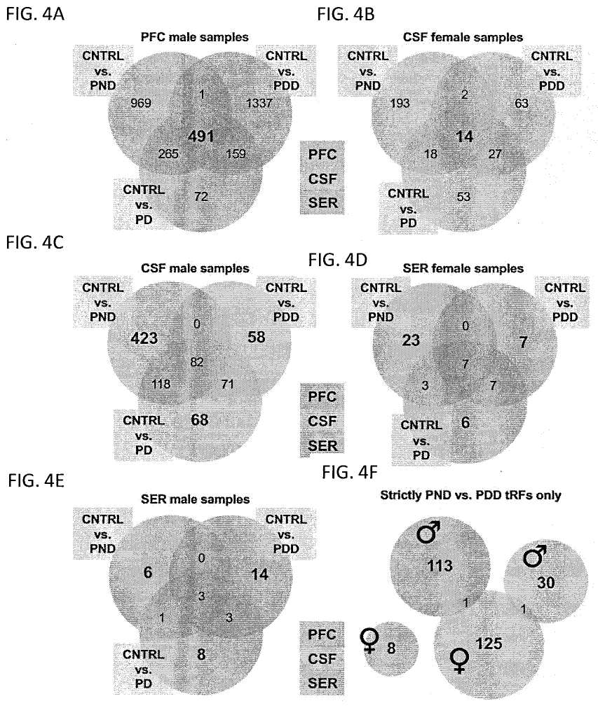

Next, tRFs that are DA between patients who had PD with dementia (PDD) and PD without dementia (PND) were sought. FIG. 4 illustrates which DA tRFs are shared among PDD, PND and CNTRL. The comparisons were carried out separately for males and females, and for each of the three collections (PFC, CSF, and SER). The DA tRFs compared here came from the {PK∪CSF∪SER}−{MINTbase} set.

Of note in PFC, it was found that 491 tRFs that are DA between CNTRL and PD, CNTRL and PND, and CNTRL and PDD patients: this indicates that these tRFs are DA independently of the stage of disease (FIG. 4A). Conversely, 265 and 159 tRFs are commonly DA between CNTRL vs. PD and CNTRL vs. PND and CNTRL vs. PDD comparisons, respectively (FIG. 4A). These tRFs likely capture changes that are specific to disease progression, and warrant further investigation into whether their roles are causative in this regard. The mutation background of these patients is not known, so it is conceivable that these tRFs could also change as a result of specific molecular alterations in individual donors.

It was also sought to determine whether any tRFs are DA between PND and PDD. It was found that a unique set of tRFs (from all five tRF structural categories) that are not contained in {MINTbase} are DA between PND and PDD, and do not appear as DA in any other comparisons (FIG. 4F). Interestingly, these select tRFs that are DA between PND and PDD show essentially no overlap among the three collections (PFC, CSF, and SER). It is necessary to emphasize that the tRFs that are DA between PND and PDD in SER exhibit a very strong dependence on the sex of the patient: males and females share only one DA tRF (FIG. 4F).

### Example 7

### tRFs Could Serve as Candidate PD Biomarkers

It was sought to determine whether DA tRFs could serve as biomarkers of PD. Two classification techniques were employed that used the abundance of tRFs as features: RPART and PLSDA (see Methods). Each RPART model was tuned to optimize the minimum number of features in each node of the decision tree. PLSDA was allowed to automatically select the number of components (latent variables) in the model. Each approach was used in a Monte Carlo simulation. The number of iterations varied across collections (see Methods). The performance assessment was done for the PLSDA model using all DA tRFs for each comparison (Table 2) and using only tRFs in {PFC∪CSF∪SER}−{MINTbase} (Table 2: “Sens” stands for sensitivity and “Spec” stands for specificity).

TABLE 2

| | Male | | Female | | Male | | Female | | Male | |
|---|---|---|---|---|---|---|---|---|---|---|
| | PFC | | CSF | | CSF | | SER | | SER | |
| | Sens | Spec | Sens | Spec | Sens | Spec | Sens | Spec | Sens | Spec |
| tRFs in | 98% | 86% | 90% | 98% | 91% | 79% | 100% | 59% | 91% | 96% |
| {PFC ∪ CSF ∪ SER} | | | | | | | | | | |
| tRFs in | 97% | 83% | 89% | 98% | 87% | 76% | 97% | 71% | 86% | 95% |
| {PFC ∪ CSF ∪ SER}- | | | | | | | | | | |
| tRHs | | | | | | | | | | |
| tRFs in | 99% | 85% | 91% | 94% | 89% | 78% | 96% | 53% | 93% | 97% |
| {PFC ∪ CSF ∪ SER}- | | | | | | | | | | |
| {MINTbase} | | | | | | | | | | |
| tRFs in | 97% | 83% | 88% | 95% | 88% | 78% | 98% | 64% | 65% | 98% |
| {PFC ∪ CSF ∪ SER}- | | | | | | | | | | |
| {MINTbase}-tRHs | | | | | | | | | | |

Also assessed, but not plotted here, are the results from using RPART, because the PLSDA model showed better performance (Table 3: “Sens” stands for sensitivity and “Spec” stands for specificity).

TABLE 3

| | Male | | Female | | Male | | Female | | Male | |
|---|---|---|---|---|---|---|---|---|---|---|
| | PFC | | CSF | | CSF | | SER | | SER | |
| | Sens | Spec | Sens | Spec | Sens | Spec | Sens | Spec | Sens | Spec |
| tRFs in | 79% | 83% | 59% | 59% | 67% | 56% | 95% | 10% | 53% | 62% |
| {PFC ∪ CSF ∪ SER} | | | | | | | | | | |
| tRFs in | 82% | 81% | 59% | 60% | 67% | 56% | 92% | 18% | 57% | 85% |
| {PFC ∪ CSF ∪ SER}- | | | | | | | | | | |
| {MINTbase} | | | | | | | | | | |

Finally, the performance of the PLSDA after scrambling the sample labels over the course of N² iterations was assessed, in order to determine how robust the PLSDA method was to noise. It was found that the performance of the model decreased dramatically, indicating that the PLSDA model identified true signal (data not shown).

Many tRNA halves have been shown to be induced by stress conditions in a wide variety of cell types. Consequently, tRNA halves are not expected to have the tissue- or disease-specificity required of good biomarker candidates. With this potential non-specificity in mind, all tRHs (5′ and 3′) were removed from the collection {PFC∪CSF∪SER}−{MINTbase}, and Monte-Carlo simulations were reran. As expected, it was observed that there was only a marginal decrease across all sensitivity and specificity measures (Table 2). Of note, there was an increase in specificity in the female SER classifier, which is anticipated given the rather small number (10) of starting tRFs (FIG. 1F).

### Example 8

### tRFs Could Serve as Candidate PND and PDD Biomarkers

Having shown the ability of tRFs to differentiate PD from CNTRL, it was sought to determine whether tRFs could also distinguish each of PDD and PND from CNTRL. In each of the comparisons, PDD vs. CNTRL and PND vs. CNTRL the DA tRFs described above and shown in FIG. 4 were used. The focus specifically was on the tRFs in {PFC∪CSF∪SER}−{MINTbase} given that these tRFs are more likely to exhibit the desired specificity that is expected of biomarkers. For the PFC samples, it was found that classification of CNTRL vs. PND and CNTRL vs. PDD is more specific than the comparison of CNTRL vs. PD (Table 4).

TABLE 4

| | Male PFC | | Female CSF | | Male CSF | | Female SER | | Male SER | |
|---|---|---|---|---|---|---|---|---|---|---|
| | Sens | Spec | Sens | Spec | Sens | Spec | Sens | Spec | Sens | Spec |
| | tRFs in {PFC ∪ CSF ∪ SER}-{MINTbase} | | | | | | | | | |
| CNTRL vs. PND | 100% | 95% | 100% | 95% | 98% | 98% | 80% | 58% | 89% | 64% |
| CNTRL vs. PDD | 100% | 99% | 100% | 99% | 98% | 93% | 98% | 78% | 57% | 68% |
| | tRFs in {PFC ∪ CSF ∪ SER}-{MINTbase}-tRHs | | | | | | | | | |
| CNTRL vs. PND | 100% | 95% | 96% | 97% | 86% | 88% | 83% | 59% | 83% | 65% |
| CNTRL vs. PDD | 100% | 98% | 99% | 92% | 89% | 83% | 98% | 77% | 37% | 59% |

Sensitivity remains at 100% across these three models, while specificity ranges from 85% to 99%. For CSF samples from female donors, the sensitivity improves from 91% to 98%. Specificity is relatively unchanged in CNTRL vs. PDD, but improves from 94% to 98% in CNTRL vs. PND. For CSF samples from male donors, sensitivity remains relatively unchanged, whereas specificity increases modestly. Curiously, sensitivity and specificity decrease substantially in SER comparisons (Table 4).

### Example 9

### Prioritizing the tRFs that Can Distinguish PD, PND, and PDD from CNTRL

Next, it was sought to prioritize the various tRFs based on their ability to discriminate PD from CNTRL. For PLSDA models using all tRFs from the original set {PFC∪CSF∪SER}, summed were the instances in which a tRF showed a Variance Importance in Projection (VIP) score≥1.5 in a PLSDA model, for all N² models (SEQ ID NOs: 57143-60526). Generally, the higher the VIP score is for any tRF, the higher the tRF's contribution to the PLSDA classification. Naturally, tRFs with higher VIP scores are comparatively better choices as biomarkers. Furthermore, the original model here to identify tRFs that were called VIP in the presence of both other potentially specific biomarkers and tRFs known to be non-specific to PD contexts were looked into.

To increase stringency of selection of highly specific biomarkers, VIP sums were filtered further keeping only those tRFs that are exclusive to the tRNA space and also absent from {MINTbase}. Notably, some of these tRFs are of the aforementioned tRH category. Even though the tRNA halves so far have been shown to be non-specific, it was opted to include these tRHs because they survived all possible filtering that is permitted by the currently available public data. As such, they can be viewed as candidate PD-specific biomarkers, with the understanding that they may lose their ‘PD-specific’ status if more data becomes available. For comparison, VIP votes for tRFs from the PLSDA models that do not include tRHs or tRFs seen in MINTbase were summer. The outcome of these steps resulted in a prioritized list of tRFs that would make ideal biomarker candidates. For example, 5′-tRFs contribute and average of 336.3 VIP votes in the analyses of the PFC collection (Table 5A).

TABLE 5A

| | tRF | # | Sum | Mean | # | Sum | Mean |
|---|---|---|---|---|---|---|---|
| Comparison | Category | tRFs | VIP | VIP | tRFs | VIP | VIP |
| Female CSF CNTRL vs. PD | Nuc i-tRF | 31 | 1755 | 56.6 | 23 | 1532 | 66.6 |
| Female CSF CNTRL vs. PD | Nuc 3′-tRF | 14 | 314 | 22.4 | 14 | 358 | 25.6 |
| Female CSF CNTRL vs. PD | Nuc 5′-tRH | 2 | 11 | 5.5 | 0 | 0 | 0.0 |
| Female CSF CNTRL vs. PD | Nuc 3′-tRH | 3 | 16 | 5.3 | 0 | 0 | 0.0 |
| Female CSF CNTRL vs. PD | Nuc 5′-tRF | 4 | 20 | 5.0 | 3 | 32 | 10.7 |
| Female CSF CNTRL vs. PD | MT i-tRF | 1 | 1 | 1.0 | 1 | 5 | 5.0 |
| Female SER CNTRL vs. PD | Nuc i-tRF | 3 | 1076 | 358.7 | 8 | 243 | 30.4 |
| Female SER CNTRL vs. PD | MT i-tRF | 1 | 19 | 19.0 | 2 | 18 | 9.0 |
| Male CSF CNTRL vs. PD | Nuc 5′-tRH | 1 | 1053 | 1053.0 | 0 | 0 | 0 |
| Male CSF CNTRL vs. PD | MT i-tRF | 3 | 1986 | 662.0 | 3 | 1524 | 508.0 |
| Male CSF CNTRL vs. PD | Nuc i-tRF | 92 | 19447 | 211.4 | 64 | 9922 | 155.0 |
| Male CSF CNTRL vs. PD | Nuc 3′-tRF | 91 | 6009 | 66.0 | 67 | 2964 | 44.2 |
| Male CSF CNTRL vs. PD | MT 3′-tRF | 1 | 42 | 42.0 | 1 | 125 | 125.0 |
| Male CSF CNTRL vs. PD | Nuc 3′-tRH | 7 | 91 | 13.0 | 0 | 0 | 0.0 |
| Male CSF CNTRL vs. PD | Nuc 5′-tRF | 2 | 8 | 4.0 | 0 | 0 | 0.0 |
| Male PFC CNTRL vs. PD | Nuc 5′-tRF | 28 | 8421 | 300.8 | 30 | 4207 | 140.2 |
| Male PFC CNTRL vs. PD | Nuc 5′-tRH | 15 | 3926 | 261.7 | 0 | 0 | 0.0 |
| Male PFC CNTRL vs. PD | MT 5′-tRF | 20 | 4663 | 233.2 | 397 | 1614 | 4.0 |
| Male PFC CNTRL vs. PD | Nuc 3′-tRF | 83 | 16322 | 196.7 | 218 | 8318 | 38.2 |
| Male PFC CNTRL vs. PD | MT 3′-tRF | 99 | 14865 | 150.2 | 86 | 7755 | 90.2 |
| Male PFC CNTRL vs. PD | Nuc i-tRF | 377 | 50092 | 132.9 | 112 | 34253 | 305.8 |
| Male PFC CNTRL vs. PD | MT i-tRF | 210 | 25153 | 119.8 | 17 | 9299 | 547.0 |
| Male PFC CNTRL vs. PD | MT 3′-tRH | 8 | 787 | 98.4 | 0 | 0 | 0 |
| Male PFC CNTRL vs. PD | Nuc 3′-tRH | 5 | 63 | 12.6 | 0 | 0 | 0 |
| Male PFC CNTRL vs. PD | MT 5′-tRH | 3 | 12 | 4.0 | 0 | 0 | 0 |
| Male SER CNTRL vs. PD | Nuc 3′-tRH | 1 | 90 | 90.0 | 0 | 0 | 0 |
| Male SER CNTRL vs. PD | Nuc i-tRF | 3 | 85 | 28.3 | 2 | 31 | 15.5 |
| Male SER CNTRL vs. PD | Nuc 3′-tRF | 4 | 39 | 9.8 | 2 | 9 | 4.5 |

When the tRFs from the other two tRF categories were subjected to the same filtering, the importance of nuclear i-tRFs as potent bi.omarkers emerged. Whereas most of the tRFs from the other categories are filtered out, nuclear i-tRFs contribute a high number of VIP votes on average in all five model contexts (Table 5A). Thus, nuclear i-tRFs may represent the most important set of tRFs to investigate in subsequent biomarker validation experiments.

It was noted that when this filtering was extended to the PLSDA models using CNTRL vs. PDD or CNTRL vs. PDD, this trend held (SEQ ID NOs:60527-63850). By design, none of the tRFs identified as VIP in these models, using the same filtering criteria of a VIP score ≥1.5, are recorded in MINTbase. Here again, i-tRFs emerge as potential PD biomarkers (Table 5B, 5C).

TABLE 5B

| | tRF | # | Sum | Mean |
|---|---|---|---|---|
| Comparison | Category | tRFs | VIP | VIP |
| Female CSF CNTRL vs. | Nuc 3′-tRF | 21 | 5237 | 249.4 |
| PDD | | | | |
| Female CSF CNTRL vs. | Nuc i-tRF | 26 | 4538 | 174.5 |
| PDD | | | | |
| Female CSF CNTRL vs. | MT i-tRF | 1 | 2 | 2.0 |
| PDD | | | | |
| Female CSF CNTRL vs. | MT 3′-tRF | 2 | 3 | 1.5 |
| PDD | | | | |
| Female CSF CNTRL vs. | Nuc i-tRF | 33 | 5345 | 162.0 |
| PND | | | | |
| Female CSF CNTRL vs. | MT i-tRF | 1 | 149 | 149.0 |
| PND | | | | |
| Female CSF CNTRL vs. | Nuc 3′-tRF | 15 | 1832 | 122.1 |
| PND | | | | |
| Female CSF CNTRL vs. | Nuc 5′-tRF | 1 | 23 | 23.0 |
| PND | | | | |
| Female SER CNTRL vs. | Nuc i-tRF | 12 | 249 | 20.8 |
| PDD | | | | |
| Female SER CNTRL vs. | Nuc 5′-tRF | 1 | 6 | 6.0 |
| PDD | | | | |
| Female SER CNTRL vs. | MT 3′-tRF | 1 | 2 | 2.0 |
| PDD | | | | |
| Female SER CNTRL vs. | Nuc 3′-tRF | 1 | 1 | 1.0 |
| PDD | | | | |
| Female SER CNTRL vs. | Nuc 3′-tRF | 4 | 7 | 1.8 |
| PND | | | | |
| Female SER CNTRL vs. | Nuc i-tRF | 3 | 4 | 1.3 |
| PND | | | | |

TABLE 5C

| | tRF | # | Sum | Mean |
|---|---|---|---|---|
| Comparison | Category | tRFs | VIP | VIP |
| Male CSF CNTRL vs. PDD | MT i-tRF | 5 | 2731 | 546.2 |
| Male CSF CNTRL vs. PDD | MT 3′-tRF | 8 | 3177 | 397.1 |
| Male CSF CNTRL vs. PDD | Nuc i-tRF | 133 | 47960 | 360.6 |
| Male CSF CNTRL vs. PDD | Nuc 3′-tRF | 245 | 11201 | 45.7 |
| Male CSF CNTRL vs. PND | Nuc i-tRF | 65 | 9603 | 147.7 |
| Male CSF CNTRL vs. PND | Nuc 3′-tRF | 49 | 5197 | 106.1 |
| Male CSF CNTRL vs. PND | MT i-tRF | 2 | 16 | 8.0 |
| Male CSF CNTRL vs. PND | Nuc 5′-tRF | 1 | 3 | 3.0 |
| Male PFC CNTRL vs. PDD | Nuc 3′-tRF | 149 | 21840 | 146.6 |
| Male PFC CNTRL vs. PDD | MT i-tRF | 373 | 48527 | 130.1 |
| Male PFC CNTRL vs. PDD | Nuc i-tRF | 679 | 79638 | 117.3 |
| Male PFC CNTRL vs. PDD | MT 5′-tRF | 18 | 862 | 47.9 |
| Male PFC CNTRL vs. PDD | Nuc 5′-tRF | 25 | 1003 | 40.1 |
| Male PFC CNTRL vs. PDD | MT 3′-tRF | 60 | 1362 | 22.7 |
| Male PFC CNTRL vs. PND | Nuc i-tRF | 520 | 57859 | 111.2 |
| Male PFC CNTRL vs. PND | Nuc 3′-tRF | 94 | 9067 | 96.5 |
| Male PFC CNTRL vs. PND | MT 5′-tRF | 25 | 1945 | 77.8 |
| Male PFC CNTRL vs. PND | MT i-tRF | 522 | 39415 | 75.5 |
| Male PFC CNTRL vs. PND | Nuc 5′-tRF | 28 | 1680 | 60.0 |
| Male PFC CNTRL vs. PND | MT 3′-tRF | 59 | 1563 | 26.5 |
| Male SER CNTRL vs. PDD | Nuc i-tRF | 1 | 1 | 1.0 |
| Male SER CNTRL vs. PND | MT 3′-tRF | 3 | 39 | 13.0 |
| Male SER CNTRL vs. PND | MT i-tRF | 1 | 4 | 4.0 |
| Male SER CNTRL vs. PND | Nuc i-tRF | 8 | 27 | 3.4 |

Though i-tRFs do not show the highest mean VIP vote in all cases, the number of unique tRFs contributing VIP votes is highest among i-FRFs in all but one case (male CSF

CNTRL vs. PDD). Additionally, although i-tRFs show the lowest mean VIP sum in female CSF CNTRL vs. PDD comparisons for example, there are 34 unique i-tRFs contributing VIP votes, as compared to only two 5′-CRFs. In all other female CNTRL vs. PDD and CNTRL vs. PND comparisons, i-tRFs exhibit among the top two mean VIP scores. In male comparisons, i-tRFs again contribute high stable means over a larger number of unique tRFs than alternative categories. Hence, i-tRFs emerge as promising biomarkers in the context of CNTRL vs. PDD or CNTRL vs. PND as well.

### Example 10

### The Candidate tRF Biomarkers are PD-specific

While the described analyses of this project were ongoing, and after Release 2.0 of MINTbase became available, several samples from Alzheimer's disease (AD) patients in NiH GEO (GSE63501) were noted. This collection includes 13 PFC samples from 3 female and 4 male control patients, and 4 female and 2 male AD patients.

To validate the specificity of PD classification signature, whether the DA tRFs with high VIP scores that emerged from the above analyses are present among these AD samples and controls was analyzed. MiNTmap was ran on the 13 samples that are part of the GSE63501 collection, and identified 5,662 tRFs with normalized abundance ≥1 RPM (SEQ NOs: 63851-69512). 3,734 of these tRFs are present in at least one female sample and 3,628 tRFs are present in at least one male control sample. 2,563 of these tRFs are present in female and 1,944 are present in male AD samples. Using the same approach and threshold settings that were employed to analyze the PFC, CSF and SER collections, 334 tRFs were identified that are DA in female control vs. female AD samples, and 8( ) tRFs that are DA in male control vs. male AD.

Finally, it was sought to identify which of the DA tRFs that can effectively distinguish PD from CNTRL are also DA between the AD and control samples of the GSE63501 collection. Any tRFs that appeared to be DA in both of these comparisons would be suboptimal choices for biomarkers, because they would not be disease-specific. On comparing the two lists, it was found that only 49 (15, respectively) of the 807 (334, respectively) tRFs that are DA between male (female, respectively) AD patients and controls are also present among the 987 tRFs that are DA between PD and CNTRL in the PFC samples. These 49+15 tRFs collectively contribute 3,924 (1.58%) of the 248,274 VIP votes in the PFC collection, i.e. a very small percentage of the total. Similarly, only 4 of the identified male AD DA tRFs are also DA in male CSF PD samples. 1 of the identified male AD DA tRFs is also DA in female CSF PD samples, and 2 of the identified male AD DA tRFs are also DA in male CSF PD samples. Together, only 1 of these 7 reidentified tRFs—a nuclear i-tRF accounting for 950 (1.40%) of 67,624 male CSF VIP votes—is among the VIP tRFs included in Table 4.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of identifying a subject in need of therapeutic intervention to treat a disease, condition, disease recurrence, or disease progression, the method comprising:
isolating fragments derived from tRNAs (tRFs) from a sample obtained from the subject; and
characterizing the tRFs and their relative abundance in the sample to identify a signature, wherein when the signature is indicative of a diagnosis of the disease, a treatment of the subject is recommended.

2. The method of claim 1, wherein the tRFs are selected from the group consisting of 3′-tRFs, 3′-tRHs, 5′-tRFs, 5′-tRHs, and i-tRFs from a mitochondrion (MT).

3. The method of claim 1, wherein the tRFs are selected from the group consisting of 3′-tRFs, 3′-tRHs, 5′-tRFs, 5′-tRHs, and i-tRFs a nucleus (Nuc).

4. The method of claim 1, wherein the sample is isolated from a cell, a tissue, an extracellular vesicle, or a body fluid obtained from the subject.

5. The method of claim 4, wherein the body fluid, the extracellular vesicle, the tissue or the cell is selected from the group consisting of bile, blood serum, plasma, cerebrospinal fluid, and prefrontal cortex.

6. The method of claim 1, wherein isolating the tRFs comprises isolating tRFs fragments with a length in the range of about 10 nucleotides to about 70 nucleotides.

7. The method of claim 1, wherein the signature comprises at least one sequence selected from the group consisting of SEQ ID NO:53135-SEQ ID NO: 63850.

8. The method of claim 1, wherein characterizing the tRFs comprises at least one assessment selected from the group consisting of sequencing the tRFs, measuring overall abundance of a tRF mapped to the genome, measuring a relative abundance of a tRF to a reference, assessing a length of a tRF, identifying starting and ending points of a tRF, identifying a genomic origin of a tRF, and identifying a terminal modification of a tRF.

9. The method of claim 1, wherein the tRFs and their relative abundance vary between the normal state as compared to disease state or condition.

10. The method of claim 1, wherein the tRFs and their relative abundance vary depending on sex of the subject.

11. The method of claim 1, wherein the disease or condition, disease recurrence, or disease progression is a brain disease.

12. The method of claim 11, wherein the brain disease is genetically predisposed.

13. The method of claim 11, wherein the brain disease is Parkinson's Disease.

14. A method of diagnosing, identifying or monitoring Parkinson's Disease (PD) in a subject in need thereof, the method comprising:
isolating tRFs from a cell obtained from the subject; quantifying the tRFs using a panel of oligonucleotides engineered to detect tRFs, or another method; analyzing levels of the tRFs present in the cell; wherein a differential in the level of measured tRFs as compared to a reference is indicative of a diagnosis or identification of PD in the subject; and
providing a treatment regimen to the subject dependent on the differential in the level of measured tRFs as compared to the reference.

15. The method of claim 14, wherein the tRFs comprise at least one sequence selected from the group consisting of SEQ ID NO:53135-SEQ ID NO: 63850.

16. A method of identifying a subject at risk for developing Parkinson's Disease (PD) or in need of therapeutic intervention to treat Parkinson's disease (PD), the method comprising:
isolating fragments of tRFs from a sample obtained from the subject; quantifying the tRFs using a panel of oligonucleotides engineered to detect tRFs, or another method; and
characterizing the tRFs and their relative abundance in the sample to identify a signature, wherein when the signature is indicative of a prognosis for developing PD or a diagnosis for PD, a treatment of the subject is recommended.

17. The method of claim 16, wherein the sample is isolated from a cell, tissue, extracellular vesicle, or body fluid obtained from the subject.

18. The method of claim 16, wherein the cell, extracellular vesicle, the tissue or the body fluid is selected from the group consisting of bile, blood serum, plasma, cerebrospinal fluid, and prefrontal cortex.

19. The method of claim 16, wherein isolating the tRFs comprises isolating tRFs with a length in the range of about 10 nucleotides to about 70 nucleotides.

20. The method of claim 16, wherein the signature comprises at least one sequence selected from the group consisting of SEQ ID NO:53135-SEQ ID NO: 63850.

21. The method of claim 16, wherein characterizing the tRFs comprises at least one assessment selected from the group consisting of sequencing tRFs, measuring overall abundance of a tRF mapped to the genome, measuring a relative abundance of a tRF to a reference, assessing a length of a tRF, identifying starting and ending points of a tRF, identifying genomic origin of a tRF, and identifying a terminal modification of a tRF.

22. The method of claim 16, wherein the tRFs and their relative abundance vary between the normal state subject as compared to a subject at a risk of or suffering from PD.

23. The method of claim 16, wherein the tRFs and their relative abundance compared to control subjects differ between subjects suffering from PD with dementia and subjects suffering from PD without dementia.

24. The method of claim 16, wherein the tRFs and their relative abundance vary with the sex of the subject.

25. The method of claim 16, wherein the tRFs and their relative abundance vary with the body fluid, extracellular vesicle, cell or tissue sample.

26. The method of claim 16, wherein the tRFs and their relative abundance vary in the prefrontal cortex samples from the normal state subjects, the subjects suffering from PD with dementia, and the subjects suffering from PD without dementia.

27. The method of claim 16, wherein the tRFs and their relative abundance vary in the cerebrospinal fluid samples from the normal state subjects, the subjects suffering from PD with dementia, and the subjects suffering from PD without dementia.

28. The method of claim 16, wherein the tRFs and their relative abundance vary in the serum samples from the normal state subjects, the subjects suffering from PD with dementia, and the subjects suffering from PD without dementia.

29. The method of claim 1, wherein the subject is a human.

30. A kit for high-throughput analysis of tRFs fragments in a sample from a subject in need thereof, the kit comprising a collection of specially-designed qPCR assays for quantitating tRFs, or a panel of engineered oligonucleotides capable of hybridizing tRFs, or another quantification method.
